(19) United States
(12) Patent Application Publication
(10) Pub. No.: US 2004/0072139 A1
(43) Pub. Date: Apr. 15, 2004

(54) Photodynamic treatment and uv-b-irradiation of a thrombocyte suspension

(76) Inventor: Harald Mohr, Hannover (DE)

Correspondence Address: Alan B Clement, Hedman & Costigan, 1185 Avenue of the Americas, New York, NY, 10036-2601, US

(21) Appl. No.: 10/332,108

(22) PCT Filed: Jul. 4, 2001
(86) PCT No.: PCT/DE01/02410

(30) Foreign Application Priority Data
Jul. 4, 2000 (DE) 100 31 851.7

(51) Int. Cl. A01N 1/02

(52) U.S. Cl. 435/2

(57) ABSTRACT

The invention relates to a method for inactivating viruses and for killing leukocytes in platelet suspensions by a combination of photodynamic treatment and UV-B irradiation.

The invention relates to a method for inactivating viruses and destroying leukocytes in platelet suspensions through a combination of photodynamic treatment and UV-B irradiation.

It is known that the therapeutic application of blood preparations involves the risk of the recipients of the blood preparation being infected with viruses. Mention may be made of, for example, the hepatitis B (HBV) and C viruses (HCV) as well as the Aids viruses HIV-1 and HIV-2. The risk is always present if no virus inactivation or virus elimination step is taken during manufacture of the preparation.

Methods for virus inactivation or virus elimination are applied to purified plasma protein concentrates such as albumin, factor VIII and factor IX preparations, so that these are in the meantime considered to be virus-safe. The virus risk of fresh plasma can at least be reduced by applying various methods. One method, for example, is quarantine storage. In this case the plasma is stored deep-frozen for three to six months and only released for use when a new blood sample from the relevant donor has been re-tested for the usual markers for HBV, HCV, HIV-1 and HIV-2 and found to be negative. Such a method cannot be used for cellular blood products such as erythrocyte and platelet concentrates since these only have a shelf life of approximately seven weeks or five days, respectively. For obvious reasons, cellular blood products also cannot be made virus-safe by solvent/detergent treatment as is possible with plasma protein concentrates and also with plasma; erythrocytes and platelets would hereby be lysed.

Intensive work is being carried out on the decontamination of cellular blood products by means of photodynamic methods. Photodynamic virus inactivation is based on illuminating the preparation concerned in solution or suspension in the presence of a photoactive substance, a photosensitiser. The irradiated light must have a wavelength which is absorbed by the photoactive substance. It is thereby activated and transfers this activation energy either directly to a substrate, which is thereby destroyed or damaged, or to oxygen molecules: activated oxygen species, i.e. oxygen radicals or singlet oxygen have a strong virucidal effect. In the favourable case, the photoactive substance used possesses a strong affinity to virus constituents, e.g. to the viral nucleic acid, and only a weak affinity to other components present in the preparation concerned. Thus, viruses are inactivated and other components are not altered. Only, one photodynamic method in accordance with European Patent 0 491 757-B1 (H. Mohr and B. Lambrecht, Process for inactivating viruses in blood and blood products) is currently in fairly wide use. It is used for inactivating viruses in fresh plasma. The phenothiazine dye methylene blue is mainly used as the photoactive substance in the technical application. Toluidine blue can also be used instead of methylene blue. The demethylation products of methylene blue, i.e. azure dyes A, B and C as well as thionine are also photodynamically active and suitable for photodynamic inactivation of viruses.

U.S. Pat. No. 5,545,516 (S. J. Wagner: Inactivation of extracellular enveloped viruses in blood and blood components by phenthiazin-5-ium dyes plus light) describes the inactivation of extracellular viruses with the aid of phenothiazine dyes combined with visible light. According to U.S. Pat. No. 5,545,516, before the photodynamic treatment leukocytes are removed from the preparations by means of special filters since the virus-inactivation method does not cover any cell-associated viruses or proviruses. This method is also incapable of inactivating small nonenveloped viruses present in the blood, such as hepatitis A viruses (HAV). However, free viruses having lipid envelopes, such as the Aids virus HIV-1 and the hepatitis B and C viruses (HBV, HCV) can be inactivated by this method. WO, 00/04930 and WO 96/08965 disclose methods for inactivating pathogens in biological samples which use photoactive substances which absorb in the UV-A range among others and are activated by radiation in the wavelength range from the UV-A to the visible.

Leukocytes in blood products can be destroyed by means of UV irradiation. For, platelet suspensions, UV-B irradiation (wavelength range 290-320 nm) has proved suitable for this purpose since an energy input of 1 to 3 J/cm² is generally sufficient for leukocyte depletion. This does not adversely influence the platelets to any significant extent, and they therefore remain therapeutically useable. An energy input higher than 10 J/cm² through UV-B irradiation additionally has a virucidal effect (K. N. Prodoux; J. C. Fratanoni, E. J. Boone and R. F. Bonner in Blood, 70(2), 589-592 (1987): Use of laser UV for inactivation of virus in blood products). However, platelets are damaged here such that their applicability must be questioned (J. C. Fratanoni and K. N. Prodoux in Transfusion 30(6), 480-481 (1990; Viral inactivation of blood products).

Thus, the object of the invention is thus to provide an effective method for inactivating human pathogenic viruses and leukocytes in platelet suspensions, especially platelet concentrates (PC). PC are obtained from blood donation by differential centrifugation or directly from donors by thrombocytapheresis. It was surprisingly found that the combination of photodynamic treatment with UV-B irradiation in platelet suspensions or platelet concentrates effectively covers the viruses accessible to photodynamic virus inactivation and at the same time is able to destroy the leukocytes present in the media, thus eliminating the risk of infection by cell-associated viruses or proviruses. Furthermore, it was surprisingly found that as a result of the combination of methods, the quantity of UV-B radiation required to destroy the leukocytes can be significantly smaller than when using UV-B irradiation alone. It was likewise surprising that the additional treatment of platelet suspensions with UV-B at an intensity which by itself has almost no virus inactivating effect significantly enhances the efficiency of the photodynamic treatment.

The method for treatment of a platelet suspension according to the invention is characterised as follows:

(A) Exposure of the suspension to radiation in a wavelength range of 400 to 750 nm, preferably 550 to 700 nm, in the presence of one or several photoactive substances which have one or several absorption maxima in the wavelength range, and

(B) Exposure of the suspension to radiation in a wavelength range of 270 to 330 nm, with an energy input of 0.1 to 10 J/cm²

wherein steps (A) and (B) can be used in arbitrary order and/or overlapping in time and in step (B) there is no substance present which can be photoactivated in the wavelength range of the radiation in accordance with step (B). Preferred embodiments are the subject matter of the dependent claims or the independent claim 13.

The platelet suspension preferably has a concentration of more than 5×10⁸ platelets per ml, especially preferably more than 10⁹/ml. The platelets can be suspended, for example, in plasma or in a platelet storage medium having an arbitrary plasma content.

Step A includes a photodynamic treatment of the platelet suspension in the presence of a photoactive substance using visible light; step B includes exposure of the preparation, the platelet-containing suspension, to light in the UV-B wavelength range. The wavelength range 270 nm to 330 nm is seen as the UV-B irradiation range in the sense of the invention.

The concentration of the photoactive substance used and energy input by illumination and UV-B irradiation are set such that any viruses present are inactivated and the leukocytes contained in the platelet suspensions are destroyed, but the efficiency of the platelets is retained.

Containers used for the treatment of the platelet suspensions are UV-B transparent containers preferably made of plastic and which can be in the form of bags, for example. However, it is also feasible to carry out the photodynamic and UV-B treatment in different containers.

It is also feasible to carry out the UV-B treatment of the platelet suspensions while the platelet suspensions are being transferred from one container to another.

The phenothiazine dyes methylene blue, azure A, B, C and thionine can be used as photoactive substances. Other photoactive substances, for example, in the concentrations known from the literature for inactivating viruses in blood products, can also be used advantageously. For phenothiazine dyes such as thionine the feasible concentration range is between approximately 0.1 and 10 μM, preferably between approximately 0.5 and 5 μM or 1 and 5 μM.

Low-pressure sodium vapour lamps whose light emission maximum occurs at 590 nm are preferably used as the light source for the photodynamic treatment, especially when thionine is used. This approximately corresponds to the absorption maximum of thionine, which is approximately 595 nm in an aqueous solution. However, other light sources are also feasible, especially if a photoactive substance is used which absorbs light in a different wavelength range to that of thionine, for example.

Special tubes, lamps or lasers emitting ultraviolet light in the wavelength range between approximately 270-330 nm, can be used for the UV-B irradiation. The energy input by the UV-B treatment can be between 0.1 and 10 J/cm², preferably between 0.3 and 6 J/cm², especially preferably between 0.5 and 3 J/cm².

### EXPERIMENTAL EXAMPLES

1. General Remarks

The experiments described hereinafter were carried out using PC which were isolated from single blood donations and suspended in blood plasma. Thionine was used as the photoactive substance. Similar results can also be achieved with other photoactive substances, for example, the phenothiazine dyes methylene blue and its derivatives azure A, B and C. The invention is merely explained by the experimental examples but is not restricted in its scope.

2. Materials and Methods

The PC used in the experiments were stored in platelet rotators for up to five days. The storage containers were commercially available PVC bags. For the photodynamic and UV-B treatment PC were transferred to polyolefin plastic bags whose film material is transparent to UV-B. An installation fined with low-pressure sodium vapour lamps was used for the illumination in the presence of thionine. The PC were illuminated from both sides. A surface emitter fitted with two UV tubes which primarily emitted UV light in the wavelength range between 290 and 320 nm was used for the UV-B irradiation.

The test virus was generally vesicular stomatitis virus (VSV), which can easily be propagated in cell culture and consequently is quantifiable in CPE assays (CPE=cytopathic effect). A number of other virus were also used in experiment 1. VSV was propagated in Vero cells. The same cells were also used for infectivity assays with which the virus titres were determined. The cell culture medium used was RPMI 1640 with 10% foetal calf serum and antibiotics. The assays were conducted in microtitre plates; The relevant samples were diluted down in one to three

steps. Eight replicas per dilution were tested. The virus titres are expressed as log₁₀TCID₅₀ (TCID=Tissue Culture Infective Doses) and were calculated as specified by Karber and Spearman (G. Kärber in Naunym-Schmiedebers Arch. Exp. Pathol. Pharmacol. 162, 480-483 (1931): Contribution to the collective treatment of pharmacological series tests; C. Spearman in Br. J. Psychol. 2, 277-282 (1908): The method of “right and wrong cases” (“constant stimuli”) without Gauss formulae).

The hypotonic shock reaction and collagen-induced aggregation were used as function tests for platelets.

Mononuclear cells were isolated from donors by means of density-gradient centrifugation. In the relevant experiments these were added to the platelet suspensions in a concentration of 5×10⁵/ml. After the photodynamic treatment or UV-B irradiation aliquots of the suspension were centrifuged at low rpm (1500 rpm for 4 min). The pelletised cells were washed three times with cell culture medium (RPMI 1640 with 10% foetal calf serum and antibiotics) and then resuspended in the same medium. The cell concentration was set at 5×10⁵/ml. For the proliferation assay the cells were stimulated with Concanavulin A (ConA, 2 μg/ml) and cultivated in 200 μl aliquots for 3-4 days at 37° C. in a CO₂ gassed incubator. The cell cultures were then added. Four hours later the BRDU incorporation rate (BRDU=bromodeoxyuridine) was determined spectrophotometrically at a wavelength of 450 nm (OD₄₅₀). The extinction values are proportional to the BRDU incorporation and thus to the viability of the cells.

Experiment 1: Inactivation of Viruses in PC by Treatment with Thionine/Light

A series of viruses were investigated to determine whether and to what extent they can be inactivated by treatment with thionine/light. The concentration of the photoactive substance was 1 μM. As shown by the results summarised in Table 1, different viruses were found to exhibit different sensitivity: thus, the model viruses for the human hepatitis-C virus BVDV and CSFV as well as the Togavirus SFV were already completely inactivated after exposure for 5 minutes, while the infectivity of VSV and SV-40 was still not completely eliminated after 30 minutes.

Experiment 2: Inactivation of VSV PC by Irradiation with UV-B

As can be seen from Table 2, VSV is highly resistant to UV-B irradiation. Even after 60 min or an energy input of approximately 20 J/cm², the virus was not yet completely inactivated. From approximately 10 min or 3 J/cm², however, the UB-B irradiation had a negative effect on functions and shelf life of platelets (not shown).

TABLE 1
Photodynamic inactivation of viruses in PC by thionine/light
treatment.

| Virus | VSV | CSFV | BVDV | SFV |
|---|---|---|---|---|
| Family | Rhabdo | Flavi | Flavi | Toga |
| Genome | SsRNA | SsRNA | ssRNA | ssRNA |
| Exposure time | 30 | 5 | 5 | 5 |
| (min) | | | | |
| Reduction of | 4.4 | ≧5.5 | ≧4.9 | ≧5.2 |
| virus titre | | | | |
| Virus | HIV-1 | SHV-1 | SV-40 | |
| Family | Retro | Herpes | Papova | |
| Genome | SsRNA | DsDNA | DsDNA | |
| Exposure time | 30 | 10 | 30 | |
| (min) | | | | |
| Reduction of | ≧5.7 | ≧3.6 | 3.9 | |
| virus titre* | | | | |

VSV = vesicular stomatitis virus;
CSFV = classical swine fever virus);
BVDV = bovine viral diarrhoea virus;
SFV = Semliki Forest virus;
HIV-1 human immunodeficiency virus, Type 1;
SHV-1 = suid herpesvirus 1;
SV-40 = simian virus-40;
ssRNA = single strand RNA;
dsDNA = double strand DNA,
*reduction of the virus titre in log₁₀TCID₅₀.

TABLE 2
Inactivation of VSV in platelet concentrates by UV-B irradiation

| UV-B | | Virus titre | Reduction factor |
|---|---|---|---|
| Min. | J/cm² | (log₁₀TCID₅₀) | (log₁₀TCID₅₀) |
| 0 | 0 | 6.44 | 0 |
| 10 | 3.25 | 5.48 | 0.96 |
| 20 | 6.5 | 4.53 | 1.91 |
| 30 | 9.75 | 4.35 | 2.09 |
| 40 | 13 | 3.28 | 3.16 |
| 50 | 16.25 | 2.33 | 4.11 |
| 60 | 19.5 | 1.61 | 4.83 |

Experiment 3: Inactivation of VSV in PC by the Combination of Thionine/Light and UV-B Radiation

In these experiments the thionine concentration was again 1

μM and the exposure time 30 min. The energy input by UV-B radiation was 2.4 J/cm² (irradiation time: 8 min). As a result of the photodynamic treatment alone, the infectivity was reduced by 4 respectively 4.2 log₁₀ and by UV-B radiation alone it was reduced by 1.97 respectively 2.21 log₁₀. When combined, the infectivity was completely eliminated in the first experiment (≧7.04 log₁₀) and reduced by 6.26 log₁₀ in the second (Table 3).

TABLE 3
Inactivation of VSV in PC by thionine/light, UV-B and the
combination of both working steps

Infectivity (log_{10L}TCID₅₀)

| Thionine/light | UV-B | Experiment 1 | Experiment 2 |
|---|---|---|---|
| − | − | 7.28 ± 0.29 | 6.68 ± 0.21 |
| + | − | 2.86 ± 0.31 | 2.68 ± 0.12 |
| − | + | 5.07 ± 0.12 | 4.71 ± 0.17 |
| + | + | ≦0.24 ± 0.00 | 0.42 ± 0.21 |

Experiment 4: Influence of Treatment with Thionine/Light Combined with UV-B on Platelet Functions.

As shown in Tables 4 and 5, neither the HSR (hypotonic shock reaction) nor the collagen-induced aggregation of platelet concentrates is substantially more strongly influenced by the combined treatment with thionine/light and UV-B (experimental conditions: see experiment 3) than by the photodynamic treatment alone.

TABLE 4
Influence of treatment of platelet suspensions with thionine/light ±
UV-B on the HSR (expressed in %) measured on day 1 and on day 3 after
treatment.

| | | Experiment 1 | | Experiment 2 | | Experiment 3 | |
|---|---|---|---|---|---|---|---|
| No. | Treatment | Day 1 | Day 3 | Day 1 | Day 3 | Day 1 | Day 3 |
| 1 | Control | 71.98 | 63.07 | 66.71 | 60.78 | 78.16 | 62.59 |
| 2 | THIONINE/light | 65.49 | 49.16 | 56.24 | 52.61 | 69.30 | 55.49 |
| 3 | UV-B (2.4 J/cm²) | 61.06 | 57.09 | 56.26 | 48.80 | 65.67 | 52.49 |
| 4 | THIONINE/Iight + UV-B | 47.86 | 51.16 | 42.37 | 30.26 | 54.45 | 48.31 |

TABLE 5
Influence of treatment of platelet suspensions with thionine/light ±
UV-B on the collagen-induced aggregation (expressed in %) measured on day
1 and on day 3 after treatment.

| | | Experiment 1 | | Experiment 2 | | Experiment 3 | |
|---|---|---|---|---|---|---|---|
| No. | Treatment | Day 1 | Day 3 | Day 1 | Day 3 | Day 1 | Day 3 |
| 1 | Control | 88.00 | 23.00 | 83.33 | 30.00 | 79.67 | 30.33 |
| 2 | Thionine/light | 73.25 | 13.75 | 84.67 | 27.67 | 69.67 | 15.67 |
| 3 | UV-B (2.4 J/cm²) | 76.25 | 11.25 | 73.33 | 24.50 | 75.67 | 20.00 |
| 4 | Thionine/light + UV-B | 69.75 | 16.75 | 76.67 | 53.50 | 58.33 | 30.33 |

Experiment 5: Inactivation of T Lymphocytes in PC by UV-B; Influence of Thionine/Light

Mononuclear cells were added to PC in a concentration of 5×10⁵/ml; they were then UV-B irradiated for various times or treated only or additionally with thionine/light (dye concentration: 2 μM; exposure time 30 min). As can be seen from the results presented in Table 6, an irradiation time of at least 4 min

(1.2 J/cm²) was required for complete inactivation of the cells. If the PC were additionally treated with thionine/light, this could be reduced to approximately 3 min although thionine/light alone had no influence on the proliferation of the cells.

TABLE 6
Inactivation of T-lymphocytes in platelet concentrates by
UV-B irradiation. Amplification of the effect by previous
treatment with Th/light for 30 min. After irradiation or Th/light
treatment, the cells were stimulated with ConA. The OD_{450 nm}
values are means of threefold determinations. They represent the
incorporation rate of BRDU in the cells after a cultivation time
of three days.

| | UV-B | | ConA- | Absorption | |
|---|---|---|---|---|---|
| No. | (J/cm²) | Th/light | activation | OD_{450 nm} | Remarks |
| 1 | 0 | 0 | − | 0.276 | Negative |
| | | | | | control |
| 2 | 0 | 0 | + | 0.269 | Positive |
| | | | | | control |
| 3 | 0.6 | − | + | 1.767 | |
| 4 | 0.9 | − | + | 0.747 | |
| 5 | 1.2 | − | + | 0.297 | |
| 6 | 0.6 | + | + | 1.020 | |
| 7 | 0.9 | + | + | 0.387 | |
| 8 | 1.2 | + | + | 0.240 | |

1. A method for treatment of a platelet suspension comprising the following steps:
(A) exposure of the suspension to radiation in a wavelength range of 400 to 750 nm in the presence of one or several photoactive substances which have one or several absorption maxima in the wavelength range, and (B) exposure of the suspension to radiation in a wavelength range of 270 to 330 nm, with an energy input of 0.1 to 10 J/cm²
wherein steps (A) and (B) can be used in arbitrary order and/or overlapping in time, and
in step (B) there is no photoactive substance present which has an absorption maximum in the wavelength range of the radiation according to step (B)

2. The method according to claim 1, characterised in that the photoactive substance is a phenothiazine dye.

3. The method according to claim 2, characterised in that thionine, methylene blue, toluidine blue and/or the azure dyes A, B and C are used as phenothiazine dyes.

4. The method according to one of claims 2 or 3, characterised in that the photoactive substance in step (A) is used in a concentration of 0.5 to 10 μM, preferably 0.5 to 5 μM.

5. The method according to one of the preceding claims, characterised in that the intensity of the radiation in step (B) is set such that the proliferation capability of T-lymphocytes contained in the platelet concentrates is reduced by at least 50%, preferably by more than 75%.

6. The method according to one of the preceding claims, characterised in that in step (B) the energy input is 0.1 to 10 J/cm², preferably 0.5 to 3 J/cm².

7. The method according to one of the preceding claims, characterised in that in step (B) the suspension is exposed to radiation in a wavelength range of 290 to 320 mm.

8. The method according to one of the preceding claims, characterised in that the platelet suspensions are platelet concentrates.

9. The method according to one of the preceding claims; characterised in that the platelet suspensions or platelet concentrates were obtained from blood donation or by platelet apheresis.

10. The method according to one of the preceding claims, characterised in that the treatment of the suspension in accordance with step (A) and/or step (B) takes place in plastic containers transparent to the appropriate radiation.

11. The method according to one of the preceding claims, characterised in that the treatment of the suspension in accordance with step (A) and/or step (B) takes place in a flow-through apparatus transparent to the appropriate radiation.

12. The method according to one of the preceding claims, characterised in that the virus concentration of the suspension is reduced by at least 5, preferably by at least 6 log₁₀ steps.

13. A method for treatment of a platelet suspension comprising the following steps:
(A) exposure of the suspension to radiation in a wavelength range of 400 to 750 nm in the presence of one or several photoactive substances which have one or several absorption maxima in the wavelength range, and
(B) exposure of the suspension to radiation in a wavelength range of 270 to 320 nm with an energy input of 0.1 to 3 J/cm²,
wherein steps (A) and (B) can be used in arbitrary order and/or overlapping in time.
